(19) United States
(12) Patent Application Publication
(10) Pub. No.: US 2010/0163043 A1
(43) Pub. Date: Jul. 1, 2010

(54) SELF-CONTAINED ORAL VENTILATION DEVICE

(76) Inventors: William T. Hart, Aptos, CA (US); Edmond A. DeFrank, Northridge, CA (US); Cesar Bandera, East Brunswick, NJ (US)

Correspondence Address: EDMOND A. DEFRANK, 20145 VIA MEDICI, NORTHRIDGE, CA, 91326, US

(21) Appl. No.: 12/492,076

(22) Filed: Jun. 25, 2009

Related U.S. Application Data
(60) Provisional application No. 61/075,344, filed on Jun. 25, 2008.

(51) Int. Cl. A61M 16/10; A61M 16/00

(52) U.S. Cl. 128/203.25; 128/204.18; 128/203.12; 128/205.12; 128/204.23

(57) ABSTRACT

The present invention is embodied in a portable self contained ventilating appliance, including an interface device coupled to at least one of an oral cavity or nasal cavity of a user, a ventilation device physically extending from the interface and configured to deliver pressurized air to the user through the interface, and a sensor monitoring system coupled to the interface and the ventilation device and configured to monitor the user.

### CROSS-REFERENCE TO RELATED APPLICATIONS

This application is based on Provisional Patent Application Ser. No. 61/075,344 filed Jun. 25, 2008, entitled “SELF-CONTAINED ORAL VENTILATION DEVICE”, by Hart et al.

### BACKGROUND

Airway occlusion during sleep occurs in individuals who suffer from sleep apnea, snoring or both. Airway occlusion during sleep produces increased airway resistance to airflow and may cause cessation of breathing and lead to hypoxemia and hypercapnia. Persons suffering from sleep apnea are at risk for systemic and pulmonary hypertension, arrhythmias leading to sudden cardiac arrest, and accidents due to hypersomnolence. Sleep apnea is most pronounced during the inspiratory phase of breathing, inhalation, and the retroglossal area is usually the most obstructed part of the airway. Airway occlusion may be caused by decline in upper airway dilator muscle tone especially of the genioglossus muscle, as well as redundant pharyngeal tissue and edema. Gravity may exacerbate occlusion of the soft tissues, which is most pronounced during the inspiratory phase of breathing. Sleeping on the back may exacerbate airway occlusion due to the added effect of gravity on the tongue and soft palate.

The dominant method of treatment for obstructive sleep apnea (OSA) is continuous positive airway pressure (CPAP). Most systems utilize an external mask over the nose and the delivery of air through an external device. Shortcomings from this system include discomfort, physiological complications, claustrophobia, travel inconvenience, and overall negative attitudes and impressions due the unsightly aesthetic appeal of nasal and full face masks.

### BRIEF SUMMARY OF THE INVENTION

The present invention provides systems, methods and devices for providing airflow with a portable self-contained ventilation device. In one embodiment, the portable self-contained ventilation device is an oral device that provides airflow through a person's mouth to their lungs for treating sleep apnea. In another embodiment, the portable self-contained ventilation device is a combination oral and nasal device that provides airflow through a person's mouth and/or nasal cavity to their lungs for treating sleep apnea.

The systems, methods and devices of one embodiment provide therapeutic treatments, health monitoring and transmit user respiratory and physiological conditions and processes to the user and health care givers. In one embodiment, the systems include analytical systems wirelessly connected to the portable self-contained ventilation device to process sensory health monitoring data gathered and perform with intervening adjustments to the air delivery treatment including distribution of therapeutic and physiological agents. The systems, methods and provide a comprehensive method for preventing airway occlusion, diagnostic, emergency and other uses in a self contained configuration that delivers treatment to the user who is no longer required to be tethered or physically connected to external equipment and gains freedom of movement while receiving the treatment.

### BRIEF DESCRIPTION OF THE DRAWINGS

FIG. 1 shows a block diagram of an overview of a method and a portable self contained ventilating appliance and an oral device for maintaining airway patency to facilitate breathing of one embodiment of the present invention.

FIG. 2 shows a flow chart of a method and appliance for preventing airway occlusion of one embodiment of the present invention.

FIG. 3A shows a block diagram of a portable self contained ventilating appliance of one embodiment of the present invention and continues on FIG. 3B.

FIG. 3B shows a block diagram of a dentally stabilizing platform apparatus of one embodiment of the present invention continued from FIG. 3A.

FIG. 4A shows for illustrative purposes only shows an example of a portable self contained ventilating appliance of one embodiment of the present invention.

FIG. 4B shows for illustrative purposes only an example a dentally stabilizing platform apparatus of one embodiment of the present invention.

FIG. 4C shows for illustrative purposes only shows an example of a portable self contained ventilating appliance and a dentally stabilizing platform apparatus connected of one embodiment of the present invention.

FIG. 5A shows for illustrative purposes only shows an example of an oral device top view of one embodiment of the present invention.

FIG. 5B shows for illustrative purposes only shows an example of an oral device perspective rear view of one embodiment of the present invention.

FIG. 5C shows for illustrative purposes only shows an example of an oral device perspective front view of one embodiment of the present invention.

FIG. 6 shows for illustrative purposes only shows a flow diagram example of an installed portable self contained ventilating appliance of one embodiment of the present invention.

FIG. 7A shows for illustrative purposes only an example of parallel oral and nasal ventilation of one embodiment of the present invention.

FIG. 7B shows for illustrative purposes only shows an example of oral ventilation assisted by nasal vacuum of one embodiment of the present invention.

FIG. 7C shows for illustrative purposes only shows an example of nasal ventilation assisted by oral vacuum of one embodiment of the present invention.

FIG. 7D shows for illustrative purposes only shows an example of ventilation assisted by percussion of one embodiment of the present invention.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview:

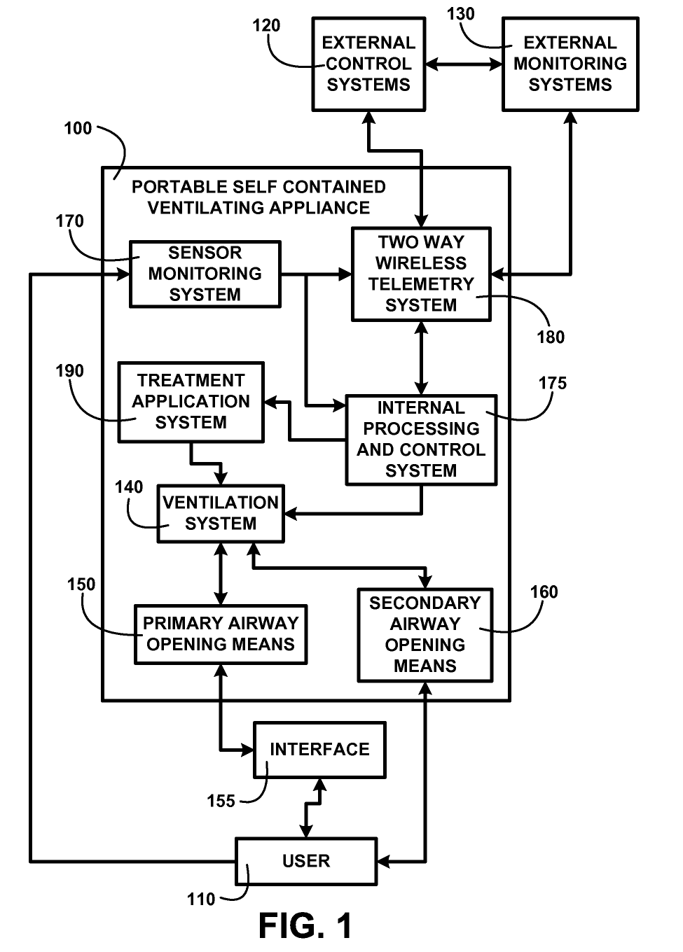

FIG. 1 shows a block diagram of an overview of a method and a portable self contained ventilating appliance 100 as an oral device for maintaining airway patency to facilitate breathing of one embodiment of the present invention. FIG. 1 shows in general, the invention as an appliance for preventing airway occlusion during sleep in individuals who suffer from sleep apnea, snoring or both. In one embodiment, the portable self-contained ventilation device 100 is an oral device that provides airflow through a person's mouth to their lungs for treating sleep apnea. In general, the oral appliance 100 prevents airway occlusion, analyzes biomarkers and physical dimensions of the airway pharynxes of individuals whether asleep or awake and maintains airway patency.

In particular, FIG. 1 shows a portable self contained ventilating appliance 100 for maintaining airway patency in a user 110 who may be experiencing airway occlusion. The portable self contained ventilating appliance 100 does not require being tethered to an external device with hoses, and thus, provides the user with mobility to move about and travel. The appliance has an internal processing and control system 175 to self adjust treatment settings internally and adjust treatment settings from external control systems 120, which can receive user condition and transmit treatment responses.

The portable self contained ventilating appliance 100 includes ventilation system 140, which performs various processes, for example filtering ambient air drawn into the system, generating pressurization of air to be delivered to the user 110. The ventilation system 140 can have a motor, fan and/or blower and delivers pressurized air to (positive pressure), or draws air from (negative pressure), the users 110 oral cavities as a primary airway opening means 150 and/or the nasal cavity as a secondary airway opening means 160. In one embodiment, the primary airway opening means 150 is connected to an interface 155, such as an oral device which is inserted into the mouth of the user 110 to maintain a passage for ambient or treated air of one embodiment of the present invention. The oral device interface 155 creates a seal with the oral cavity using various methods, for example, a dentally stabilized platform apparatus or inflatable seals. In another embodiment, the portable self contained ventilating appliance 100 is connected to a CPAP nasal mask or a CPAP full face mask.

The portable self contained ventilating appliance 100 includes sensor monitoring system 170, which for example measures parameters of respiration. The information obtained through the sensor monitoring system 170 is transmitted to the internal processing and control system 175. The internal processing and control system 175 can self adjust treatment settings in response to information obtained through the sensor monitoring system 170 according to programmable parameters. The internal processing and control system 175 can transmit sensor monitoring system 170 information and current settings through a two way wireless telemetry system 180 contained within the portable self contained ventilating appliance 100 to external monitoring systems 130 for example a hospital, doctors office, care giver or the user themselves.

The sensor monitoring system 170 information can also be transmitted through the two way wireless telemetry system 180 to the external control systems 120 and other devices for example computer systems, blue tooth communication devices, cell phones, WIFI or other data or communication devices. The transmission of user responses using the two way wireless telemetry system 180 and external control systems 120 provides a diagnostic means for the user 110 or monitoring health care persons to assess the user condition and response to the treatment. It provides a method in which the user 110 or monitoring health care persons can initiate changes in the settings of the portable self contained ventilating appliance 100 including activation of treatment application system 190 to administer for example medications to relieve a particular symptom. The user response data collected by the sensor monitoring system 170 and operating status of the portable self contained ventilating appliance 100 can be transmitted to a data storage collection system in the external control systems 120 for current or later use for example a review of user 110 compliance or history of use of the treatment, progress of treatment responses or other assessment purposes of one embodiment of the present invention.

### Detailed Operation:

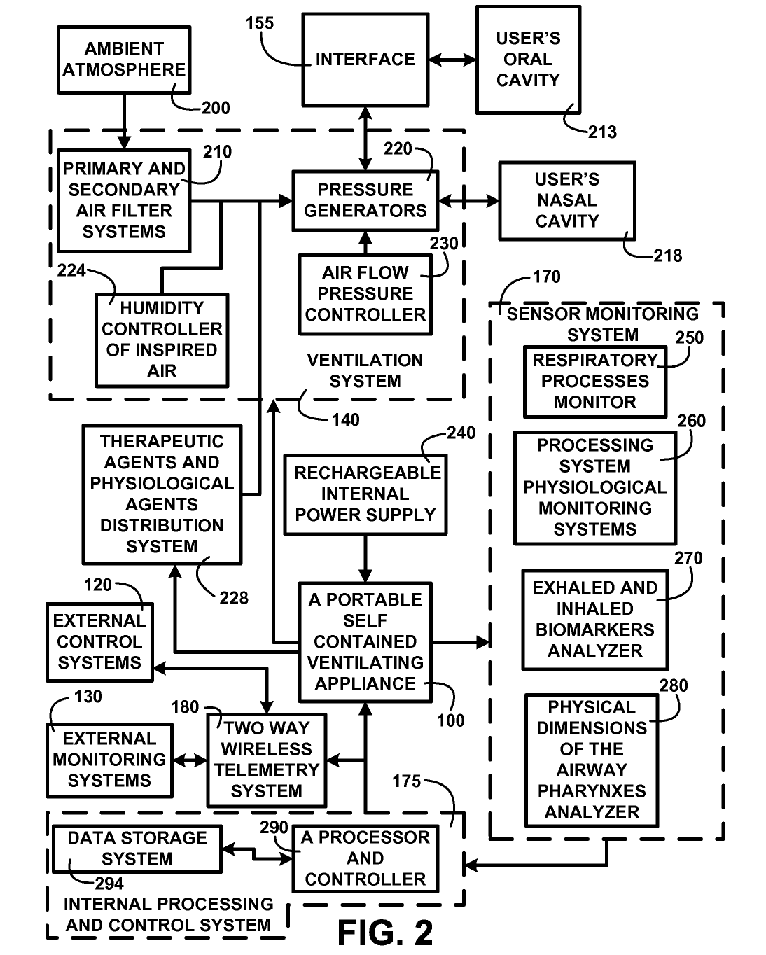

FIG. 2 shows a flow chart of a method and appliance for preventing airway occlusion of one embodiment of the present invention. FIG. 2 shows a flow chart of a method for preventing airway occlusion through the use of the portable self contained ventilating appliance 100 and interface, such as an oral device 155.

The portable self contained ventilating appliance 100 uses a rechargeable internal power supply 240 to power the miniaturized systems within the appliance. Used in concert with the portable self contained ventilating appliance 100 is the interface device 155 which is connected to the appliance 100. In one embodiment, a oral device is used and inserted into a user's oral cavity. One element of the ventilation system 140 is pressure generators 220 in the appliance which create both positive and negative pressure that can be applied separately or in combination to the user's oral cavity 213 and/or user's nasal cavity 218.

### Air Filter Systems:

Air is drawn from the ambient atmosphere 200 and passes through primary and secondary air filter systems 210 into the appliance. The filtering systems prevent raw ambient air from entering the user's airway. In one embodiment, the air filter system is used with oral, nasal and/or full face masks. With the masks, clean air is forced into an interior gas space through an air filter. The wearer breathes this clean air and exhales it back into the breathing zone. This exhaled air exits through openings in a face seal precluding contaminants from entering the interior gas space through the openings. Other embodiments include a variety of face seal constructions for providing a separation barrier between the interior and the exterior gas space. These products also have incorporated an elastic material around the edge of the face seal to enable it to conform to different shaped faces. A face seal prevents leaking of ambient air into the nasal and/or oral airways. The present invention provides face seals that can manage air flow within the interior gas space while protecting the wearer from contaminant inflow.

The present invention would have disposable filters for the various contaminants. When contaminated the filters would have an indicator to determine their efficacy for further or continued use. The tubular feature has a filtering feature that may be disposable, and has the capacity to attach to a variety of filters that may be applicable to the removal of particulate material or chemical agents in the ambient air. Ambient air is breathed into the oral appliance through an air filter. The wearer breathes this clean air and exhales it back through the breathing zone. This exhaled air exits through openings in the face seal precluding contaminants from entering the interior gas space through the openings. The rhinal seal provides a separation barrier between the interior and the exterior gas space. A nasal seal for example can be incorporated to ensure nasal and/or oral airways are precluded from either chemical or particulate contaminants.

In one embodiment, a supplied air helmet is included that comprises a visor and a face seal where the face seal is secured to the visor and includes a sealing member. The sealing member extends there from radially inward and has at least first and second regions of permeability, wherein the first region has greater permeability than the second region. The air helmet is beneficial in that use of a face seal with differentiated permeability provides a controlled flow of air within the interior gas space. Airflow can be managed, for example, to allow it to flow from an inlet port located at the wearer's forehead, across the front of the wearer's face, and then exit the interior gas space beneath the chin. Air that exits the interior gas space can readily enter the exterior gas space to allow room for new clean air to be furnished to the breathing zone. The managed airflow provides a comfortable environment to the wearer and may also provide less opportunity for the wearer to inhale contaminants that may be present in the ambient environment or exterior gas space.

### Air Flow Pressure Controller:

An air flow pressure controller 230 regulates the applications of positive and negative pressure to the user's oral cavity 214 and or user's nasal cavity 218. Sensor monitoring system 170 within the portable self contained ventilating appliance 100 and the oral device 155 gather information for example user respiratory and other physiological processes and status.

### Data Gathering, Processing And Storage System:

The gathered information is transmitted to the internal processing and control system 175 which includes a processor and controller 290 and to a data storage system 294. The ventilating appliance has an internal processing and control system 175. The processing correlates the information from the sensory system, the analyzed information, and the distribution system to produce desired components to be added to the respiratory airflow. This processing system monitors and continually adjusts the airflow in relation to the biomarkers being analyzed. The mechanical feature contains a data storage and data recording systems for the maintenance of a record of exhaled biomarkers and the calculation of agents to be added to the respiratory flow through the appliance.

### Sensory Systems:

The information is obtained through a series of sensor monitoring system 170. Sensory system using sensors for the purpose of analyzing physiological parameters associated with the provision and maintenance of airflow as well as for the measurement of those parameters that indicate changes in the physiological condition of the user. One embodiment includes a sensor tube extending through the user's teeth and lips, for connection to an air pressure monitor which controls the negative air supply.

### Respiratory Processes Monitor:

One sensory system is operated by a respiratory processes monitor 250. This sensory monitor provides a method of monitoring parameters of the user's respiratory processes, and a processor and controller capable of collecting, interpreting, and acting on information regarding those parameters. This respiratory information is used in activating controlling systems to respond to changes in the user's breathing.

In one embodiment a light-weight monitor device incorporates physiological sensors, including a pulse oximeter to detect oximetry and pulse rate, a microphone to detect snoring sounds, and a position sensor to detect patient head position. In another embodiment, the system may be modified to measure airflow with a thermistor or a plastic cannula running from the enclosure across the bridge of the patient's nose to the nares. The monitor device contains a power source and a digital memory system to store or record the monitored signals and data.

### Physiological Monitoring:

Another sensory and analysis system is a processing system physiological monitoring systems 260. The physiological monitoring system may also contain additional sensors or may use the same sensors to monitor other physiological signals, such as ocular movement or heart rate. The physiological monitoring system may also interface or be integrated with a neuromuscular stimulation device, thereby eliminating the need for an external controller device, e. g., a bedside controller.

In another embodiment, a physiological monitoring system may be used for monitoring SaO₂ (arterial oxygen saturation) in patients in real-time with a digital display LCD incorporated into the monitoring device to present the SaO₂ values to the patient. An exhaled and inhaled biomarkers analyzer 270 gathers information on biomarkers. The assessment of biomarkers is important in the determination of the causation and treatment of diseases including respiratory diseases such as asthma and chronic obstructive pulmonary disease (COPD). Biomarkers may be objectively measured and the measures compared to those parameters normally expected from those biological processes, pathological conditions, or the biological response to therapeutic intervention.

The present invention includes a processing system that provides systems and methods for physiological monitoring of a patient. In one embodiment, the hollow body of the oral appliance incorporates physiological sensors, including but not limited to, a pulse oximeter, heart rate, a microphone to detect snoring sounds, airflow, temperature, oxygen, nitric oxide, carbon dioxide, minute volume, tidal volume, gas pressure, etc.

The monitor device contains a power source and memory to store or record the monitored signals and data. The physiological monitoring system may be integrated with other therapeutic devices, such as a Continuous Positive Airway Pressure (CPAP) device. The physiological monitoring system may also interface or be integrated with a neuromuscular stimulation device.

### Physical Dimensions Analyzer:

An analysis system using a physical dimensions of the airway pharynxes analyzer 280 uses sensors and a set of parameters to determine physical changes in the airway pharynxes. As this information is processed it can signal a humidity controller of inspired air 224 to adjust the amount of humidity.

### Humidity Controller:

This invention also relates to effectively controlling the humidity of inspired air. The delivery of the humidified air would be synchronous with the user's breathing cycle. Such systems would decrease the sensation of dryness for the user, and increase comfort and compliance.

A feature of the system is the creation of aerosolized water droplets, introducing said droplets into the breathing circuit of a respiratory therapy apparatus for delivery to the user's airway. Such droplets could be controlled for size and velocity as well as rate of delivery to specific segments of the user's airway. The apparatus would be external to the oral appliance. The feature would be to effectively control the amount and location of moisture delivered to the user's airway in response to a parameter/s of respiration, which may include, flow rate, tidal volume, minute volume, pressure, vapor pressure of exhaled gas, and temperature of gas in the breathing circuit of the user's airway. The introduction of said aerosolized droplets into said airway corresponds to the inspiratory phase of the user. To minimize the collection of moisture in the breathing circuit of the respiratory breathing apparatus it may be under the control of the user.

To minimize the collection of moisture in the breathing circuit of the respiratory breathing apparatus it may be under the control of the user. In one embodiment, the apparatus utilizes the Bernoulli principle to draw fluid from the saliva to the air flowing through the apparatus. This collection of fluid may be increased by another embodiment of the invention that requires a multiple of fenestrations that are able to wick fluid to the area of air flow.

In another embodiment the present invention the oral appliance has a feature to moisturize the airway during respiratory therapy. This feature would decrease the sensation of dryness for the user, and increase comfort and compliance. The apparatus would be self contained in the oral appliance and would provide flexibility of use. A further aspect of the feature would be to effectively control the amount and location of moisture delivered to the user's airway. The process would minimize the collection of moisture in the breathing circuit of the respiratory breathing apparatus and could be delivered in conjunction with positive/negative airway treatment therapy. The feature would be to effectively control the amount and location of moisture delivered to the user's airway in response to a parameter/s of respiration, which may include, flow rate, tidal volume, minute volume, pressure, vapor pressure of exhaled gas, and temperature of gas in the breathing circuit of the user's airway. The introduction of said aerosolized droplets into said airway corresponds to the inspiratory phase of the user.

### Treatment Application System:

The treatment application system 190 of FIG. 1 includes a method for the delivery of pharmacologically active agents to the oral/respiratory mucosa comprising; generating aerosolized droplets containing or comprised of a pharmacologically active agent/s; introducing said droplets into the flow circuit of a respiratory therapy apparatus for delivery to a patient's airway wherein said respiratory therapy apparatus comprises a flow generating device, an oral appliance and an intervening flow circuit. Such agents may be for the purpose of treating obstructive sleep apnea, increasing muscle tone in the retroglossal airway, and may include acetyl choline inhibitors.

The present invention including the oral appliance has a feature for the storage of various agents including pharmacologically active agents to be delivered to the oral/respiratory mucosa. Aerosolized droplets containing or comprised of a pharmacologically active agent/s; introducing said droplets into the flow circuit of the respiratory apparatus for delivery to a patient's airway wherein said respiratory therapy apparatus comprises a flow generating device, within the oral appliance and an intervening flow circuit. Such agents may be for the purpose of treating obstructive sleep apnea, increasing muscle tone in the retroglossal airway, and may include acetyl choline inhibitors and such other agents for the control of disease and physiological function.

The gathered information can also activate a therapeutic agents and physiological agents distribution system 228 to dispense treatments. The therapeutic agents and physiological agents distribution system 228 uses methods, devices and kits for dispensing treatments via the airflow through a person's mouth to their airway. A feature is the distribution of therapeutic agents, medications, therapeutic gases (such as, but not limited to oxygen, Nitric Oxide, Helium), diagnostic imaging agents, and anesthetic gases.

The therapeutic agents and physiological agents distribution system 228 can dispense for example treatments such as therapeutic agents, medications, therapeutic gases for example oxygen, Nitric Oxide, Helium, diagnostic imaging agents, and anesthetic gases. The system would include a method for the delivery of pharmacologically active agents to the oral/respiratory mucosa comprising; generating aerosolized droplets containing or comprised of a pharmacologically active agent/s; introducing said droplets into the flow circuit of a respiratory therapy apparatus for delivery to a patient's airway wherein said respiratory therapy apparatus comprises a flow generating device, an oral appliance and an intervening flow circuit. Such agents may be for the purpose of treating obstructive sleep apnea, increasing muscle tone in the retroglossal airway, and may include acetyl choline inhibitors, as well as other agents for the control or treatment of respiratory disease or malfunction.

### Wireless Telemetry and Biotelemetry System:

The information can also be processed to a two way wireless telemetry system 180 built into the portable self contained ventilating appliance 100. This technology allows the gathered information to be shared with external monitoring devices 130 which can interact with the appliance. A telemetry system provides access to external monitoring devices 130 for example external processors and data regarding the patient, and may be used in conjunction with the embedded processors and data of the invention, to determine the status of the patient, record and process data from the patient, and provide interventions to administer agents that would result in a change in status of the patient. Alternatively, a low-powered radio frequency transmitter can be used to provide wireless transmission between the monitor and a bedside unit that displays the SaO₂ results. In either of these real-time applications, the need for onboard memory will be reduced or eliminated. Data storage systems, methods and devices for the storage of sleep apnea data can assist in medical assessment on treatment effectiveness.

In another embodiment the oral appliance has an internal sensory system linked to an external monitoring system or systems by a biotelemetry system contained in the appliance. This system may respond to external controllers for the analysis of biomarkers or the distribution of agents into the airflow. This system also has an alarm and contact component for connecting to external contacts. The system may relate data to other clinical outcomes, the progression of a disease, the severity of a disease, response to therapy, the prediction of disease, prediction of disease progression, disease instability, and the prediction of response to therapy including those therapies being developed. In one embodiment, the system would relay processed data via wireless transmission between a patient user and a central unit that displays results in either real-time or maintained for later retrieval.

### Health Monitoring System:

The present invention in addition to therapeutic treatment includes a health monitoring system including a database and data management system linked with a plurality of health trackers, each of which regularly collects various forms of data about or from a patient user. The health monitoring system for example three components a data management system including the database; a plurality of physiological and subjective data collection devices that collect a set of time stamped serial streams from a subject; and a communications system by which the data is periodically uploaded from the monitors to the database.

Data gathered from the sensory systems can be sourced from for example one or more of the some or all of the following sources: Physiological data from the patient collected from O-Vent sensors (quantity of time used/compliance, number of apnea events) as well as other adjacently attached sensors to establish sleep quality/quantity. Data could include Slow Wave Sleep or EEG data, pulse oximetry, eye movement, limb movement, metabolic data, etc. and transmitted wirelessly to a smart cell phone and/or a Web-enabled PDA (DEVICE) such as Blackberry or iPhone.

A proprietary ‘game’ that can be played in <10 minutes by the user on a device to assess neuro human performance validated against established Psychomotor Vigilance Task (PVT) and/or the Probed Recall Memory (PRM), and Iowa Gambling Task (IGT) tests. Sleep deprivation is an antecedent to declines in PVT, PRM, and IGT are positively and significantly correlated with lower work productivity as measured by established tools such as the Endicott Work Productivity Scale (EWPS) and risk-taking behavior.

A proprietary 1-3 minute recording of a user reading out loud a passage displayed on a device that detects speech changes through a proprietary algorithm validated against established methods for detecting intelligible and typical speaking styles associated with sleep deprivation. The algorithm will be validated against established methods. The data can be analyzed via proprietary software on the device and an output textually and graphically displayed for the user. Data can be transmitted from the device to a file server (via either cellular, web or other wired or wireless methods) for storage and retrieval by the user and other stakeholders. The utility is to provide data that can be used by users and stakeholders as an input to help determine if a user may be impaired in performance because of sub-optimal sleep.

In another embodiment an algorithm and associated computer software that analyzes user-sourced data to create an output value to measure neurobehavioral functionality and alertness after a rest/sleep period. The output value can provide the user and other stakeholders (employers, etc.) with information on whether or not the user may be impaired at certain tasks because of suboptimal sleep quality and quantity.

In one embodiment a portable monitor having the capability to constrict, manage; and store a detailed, multi-parametric, record of an individual's physiological and emotional wellbeing is used for tracking and assessing general health over days, months, and years. The present invention comprises a health monitoring system including a database and data management system linked with a plurality of health trackers, each of which regularly collects various forms of data about or from a patient/subject.

In one embodiment a monitoring system consists of three basic components: 1) a data management system including the database; 2) a plurality of physiological and subjective data collection devices that collect a set of time stamped serial streams from a subject; and 3) a communications system by which the data is periodically uploaded from the monitors to the database. Biotelemetry system apparatus for detecting, receiving, deriving and displaying human physiological and contextual information A monitoring apparatus that includes a sensor device and an I/O device in communication with the sensor device that generates derived data using the data from the sensor device. The sensor device includes a processor programmed to generate data sensor data. Alternatively, the present invention includes an apparatus for tracking information for an individual that utilizes a plurality of classification identifiers. Systems and methods are provided for medical monitoring of data with a transport medium external to the patient premises. A processor in communication with the application devices is adapted to coordinate transmission of the collected medical data over the transport medium external to the patient premises. The present invention in one embodiment provides systems, methods, devices and kits for providing airflow through a person's mouth to their airway.

### Portable Self Contained Ventilating Appliance:

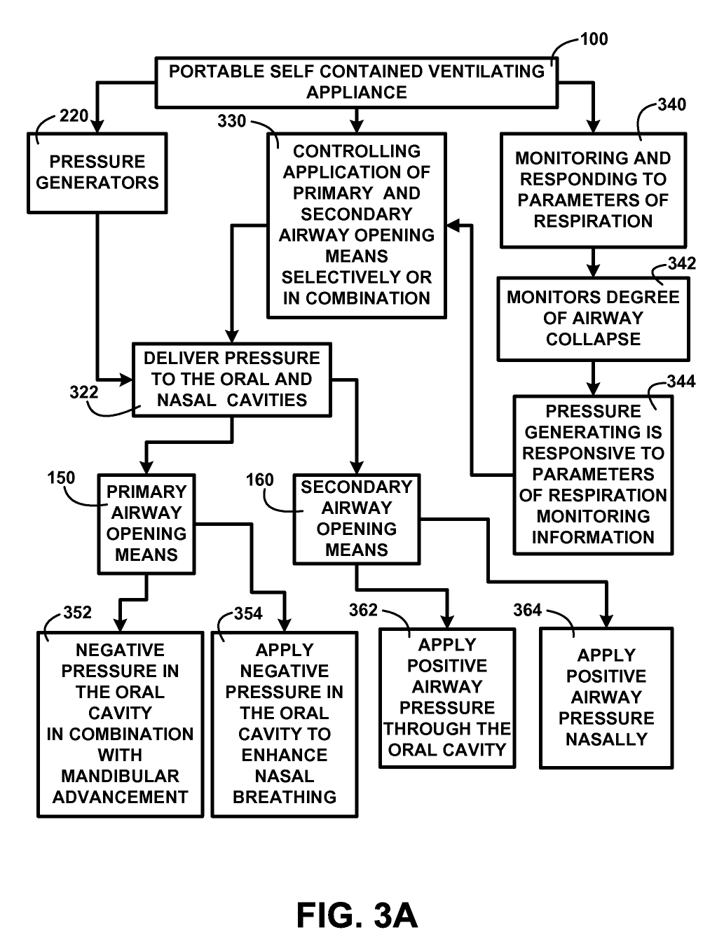

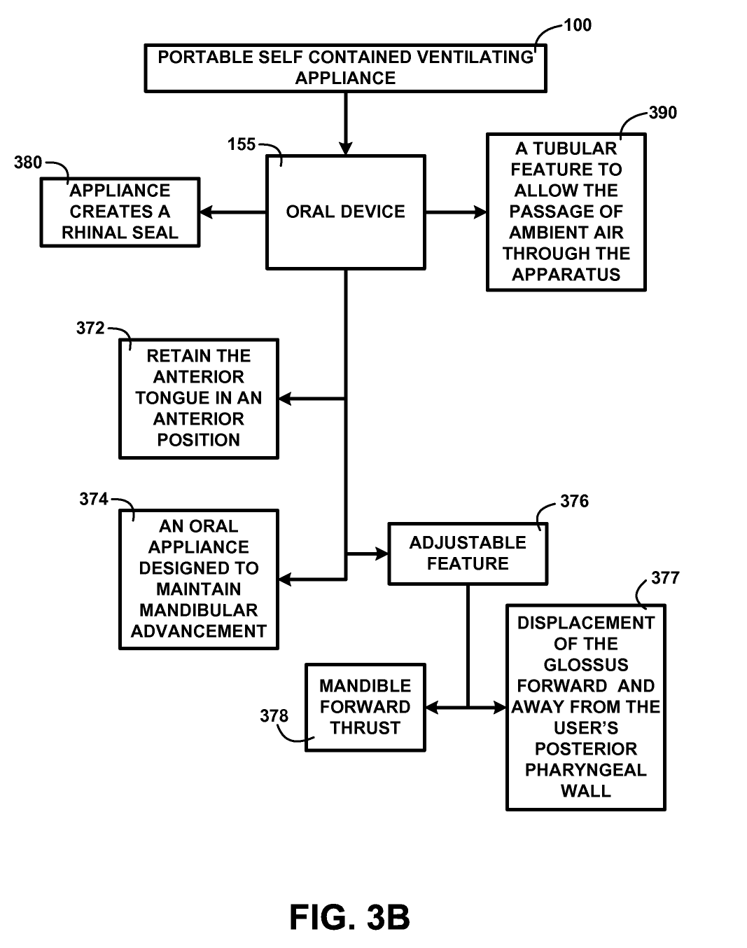

FIG. 3A shows a block diagram of a portable self contained ventilating appliance of one embodiment of the present invention and continues on FIG. 3B. FIG. 3A shows the method for maintaining airway patency which is performing non-invasive ventilation uses the portable self contained ventilating appliance 100. The portable self contained ventilating appliance 100 has pressure generators 220 to deliver pressure to the oral and nasal cavities 322. Pressure delivered to the oral cavity is an element of a primary airway opening means 150. An example of airway opening operation is by negative pressure in the oral cavity in combination with mandilbular advancement 352. Another example is to apply negative pressure in the oral cavity to enhance nasal breathing 354. Pressure delivered to the nasal cavity is an element of a secondary airway opening means 160. Some examples of airway opening operations are to apply positive airway pressure through the oral cavity 362 and to apply positive airway pressure nasally 364.

The portable self contained ventilating appliance 100 is an appliance for preventing airway occlusion during sleep in individuals who suffer from sleep apnea, snoring or both. This appliance also is used for analyzing biomarkers and physical dimensions of the airway pharynxes of individuals whether asleep or awake. More particularly this appliance is inserted into a user's oral cavity. One embodiment utilizes airway opening means selectively or in combination. The primary airway opening means will maximize user comfort and to facilitate normal breathing. Additional airway means would provide maximum efficacy in maintaining or restoring airway patency.

This appliance is used for treatment utilizing the primary airway means until such time as the level of efficacy is insufficient to maintain airway patency, after which time the complimentary airway means either replaces, or works in concert, with the primary airway means, to ensure airway patency is maintained. An embodiment of this invention incorporates primary and secondary air filter systems to clean the ambient air being drawn in by the portable self contained ventilating appliance 100.

The portable self contained ventilating appliance 100 actively adjusts and regulates delivery of pressure to the primary and secondary airway opening means. These adjustments are made by monitoring and responding to parameters of respiration 340. The system also monitors degree of airway collapse 342. The pressure generating is responsive to parameters of respiration monitoring information 344. The systems for controlling application of primary and secondary airway opening means selectively or in combination 330 help maintain airway patency. The systems allow for repeated sampling and in time measurement of response and the need for further intervention. The systems include in a method of monitoring parameters of the user's respiratory processes, a processor and controller capable of collecting, interpreting, and acting on information regarding those parameters.

The portable self contained ventilating appliance 100 also detects exhaled and inhaled biomarkers. Objective measures of physiological responses to inhaled agents as well as measures of exhaled biomarkers to evaluate the need for intervention are made. Some of the interventions may use for example a method of distributing therapeutic agents and physiological agents. The present invention includes a method for the storage of respiratory and other physiological data, and to the storage of data regarding the administration of therapeutic agents, and recording the measurement and of those agents distributed in the airway. In other embodiments with the oral appliance, a gyroscope or balanced rotor or motor to prevent vibration can be integrated with the device.

The appliance includes a telemetry system which provides access to external processors and data regarding the patient, and may be used in conjunction with the embedded processors and data of the appliance, to determine the status of the patient, record and process data from the patient, and provide interventions to administer agents that would result in a change in status of the patient.

In one embodiment the appliance is used for preventing airway occlusion during sleep including individuals presenting with sleep apnea or snoring with air flow positive and negative pressure. The particular portion of the appliance located in the oral cavity contains a mechanical feature for the increase or decrease of airflow providing airflow through a person's mouth to their airway. The present invention can also be used in providing ventilation during emergency care, or during surgery. The present invention minimizes side affects associated with positive airway pressure, including, nasal drying, air ingestion, epitasis, chest wall discomfort and other side effects.

Nasal breathing may also be facilitated by applying negative pressure to the oral cavity, so as to draw the soft palate and associated tissues, and the posterior tongue anteriorly, and together forming a seal or partial seal, thereby maintaining patency or increasing the cross sectional area of the nasopharyngeal, oropharyngeal or retroglossal airway. To facilitate nasal breathing the following steps are required. Firstly a negative pressure is applied to the oral cavity so as to draw the soft palate and associated tissues and the posterior tongue forward and together forming a seal or partial seal, and then delivering a positive pressure to the nasal airway. In an embodiment of one invention, a hollow body, conforming to the user's mouth has an opening at the rear through which a partial vacuum may be drawn in the user's oral cavity. This hollow body is the dentally stabilized platform apparatus 150 which is described in FIG. 3B of one embodiment of the present invention.

### Dentally Stabilizing Platform Apparatus:

FIG. 3B shows a block diagram of a dentally stabilizing platform apparatus of one embodiment of the present invention continued from FIG. 3A. FIG. 3B shows the method for maintaining airway patency which is performing non-invasive ventilation uses the portable self contained ventilating appliance 100 described in FIG. 3A with the oral device 155 connected.

The structure of the oral device 155 will retain the anterior tongue in an anterior position 372. Also, a retractable arm or retractable stent or lever or balloon can extends from the oral device to physically move the tongue from the airway. Moreover, application of transcutaneous nerve stimulation of the tongue muscle by the appliance to cause the tongue to move and open the airway can be implemented.

This apparatus is an oral appliance designed to maintain mandilbular advancement 374 when inserted into the user's oral cavity 130 of FIG. 2. The oral device 155 includes an adjustable feature 376 to promote mandible forward thrust 378 and the displacement of the glossus forward and away from the user's posterior pharyngeal wall 377. The physical feature of the appliance creates a rhinal seal 380 and includes a tubular feature to allow the passage of ambient air through the apparatus 390 of one embodiment of the present invention.

### Example Of A Portable Self Contained Ventilating Appliance:

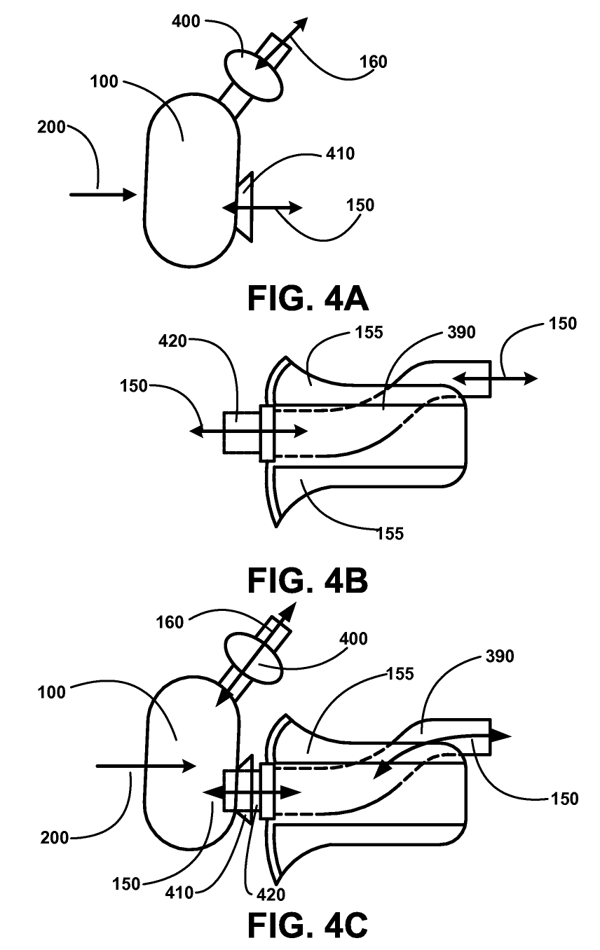

FIG. 4A shows for illustrative purposes only shows an example of a portable self contained ventilating appliance of one embodiment of the present invention. FIG. 4A shows an illustration of one example of the portable self contained ventilating appliance 100. The ambient atmosphere 200 is drawn, filtered and forced or drawn through a nasal airway connector 400 to form a part of the secondary airway opening means 160. Air can be forced or drawn through the dentally stabilized platform connector 410 to form a part of the primary airway opening means 150 of one embodiment of the present invention.

### Example of a Dentally Stabilizing Platform Apparatus:

FIG. 4B shows for illustrative purposes only an example of a dentally stabilizing platform apparatus of one embodiment of the present invention. FIG. 4B illustrates one example of the oral device 155 which has the tubular feature to allow the passage of ambient air through the apparatus 390. Air is pressure forced or drawn through a ventilating appliance connector 420 to from the primary airway opening means 150 of one embodiment of the present invention.

### Example Of A Ventilating Appliance And A Platform Apparatus Connected:

FIG. 4C shows for illustrative purposes only shows an example of a portable self contained ventilating appliance and a dentally stabilizing platform apparatus connected of one embodiment of the present invention. The FIG. 4C illustration shows one example of how the portable self contained ventilating appliance 100 and the oral device 155 are connected. This combination of the appliance and apparatus form the primary airway opening means and a secondary airway opening means which are utilized in concert to facilitate breathing as part of the method for maintaining airway patency in the present invention.

The ambient atmosphere 200 is drawn into the portable self contained ventilating appliance 100 where it is filtered. This filtered air is pressure forced through a nasal airway connector 400 to be used as a positive pressure for the secondary airway opening means 160 or is drawn through the nasal airway connector 400 in creating negative pressure in the nasal cavity of one embodiment of the present invention.

Filtered air is pressure forced through the dentally stabilized platform connector 410 to the ventilating appliance connector 420 of the oral device 155. The air passes through the tubular feature to allow the passage of ambient air through the apparatus 390 to form the positive pressure of the primary airway opening means 150. Air can be drawn by the portable self contained ventilating appliance 100 through the tubular feature to allow the passage of ambient air through the apparatus 390 to create the negative pressure used in the primary airway opening means 150 of one embodiment of the present invention.

### Example of an Oral Device:

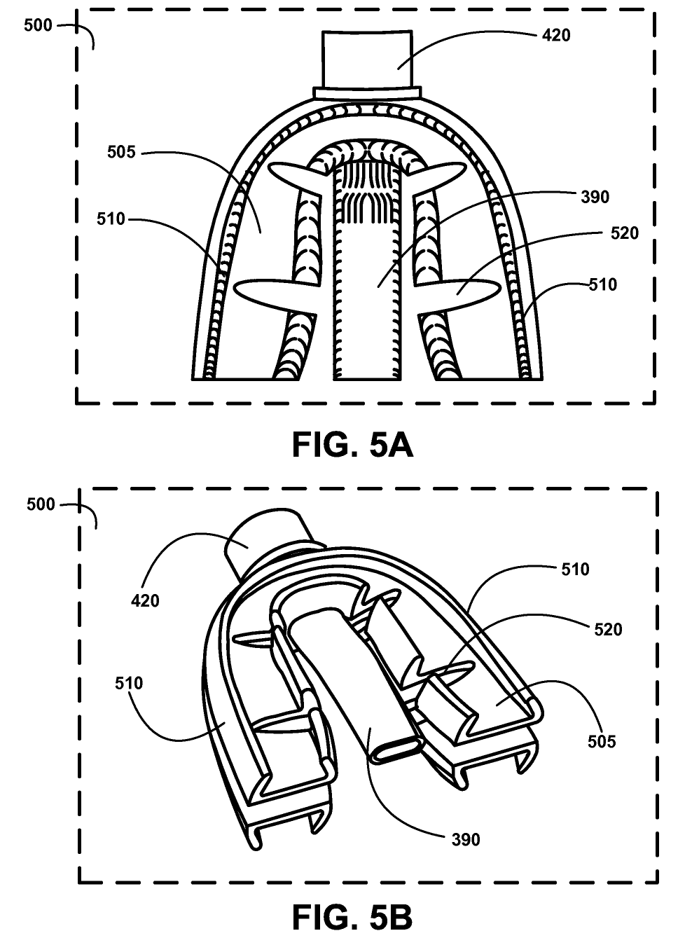

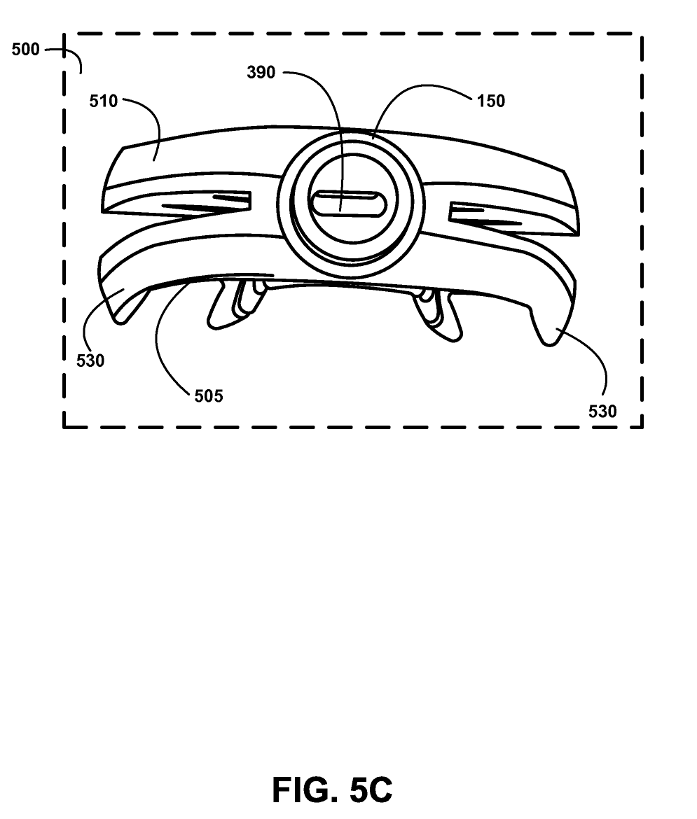

FIG. 5A shows for illustrative purposes only shows an example of an oral device top view of one embodiment of the present invention. A dentally stabilized platform apparatus 500 shown in FIG. 5A is one example of the oral device 155 of FIG. 1. The tubular feature to allow the passage of ambient air through the apparatus 390 is shown starting from the tube connector at the front and terminating at the rear. A tooth channel 505 follows the curved shape of the dentally stabilized platform apparatus 500. An intra-oral sealing flange 510 located on both sides of the tooth channel 505 creates a seal with the lips and teeth. A sealing gasket is attached to the outside of a tooth channel to which, when in-situ, a vacuum is applied to seal the cheek or lips to the gasket thus resulting in a seal between the device and the cheeks or lips. In this case the gasket cushions the tissue at the seal location for comfort and to prevent tissue irritation. The compliant gasket material also facilitates the ability for the vacuum to create and maintain a seal, as the gasket fills in the crevices and undulating shapes at the seal locations. The half elliptically shaped flexing wing cut outs 520 provide flexibility in the apparatus for comfort of one embodiment of the present invention.

FIG. 5B shows for illustrative purposes only shows an example of an oral device perspective rear view of one embodiment of the present invention. FIG. 5B provides through an illustration a view of one example of the tubular feature to allow the passage of ambient air through the apparatus 390 with its arching form extending to the rear of the dentally stabilized platform apparatus 500 which is an example of the oral device 155 of FIG. 1. This prospective shows the mirrored positioning of both the upper and lower tooth channel 505 which are bounded by the inside and outside intra-oral sealing flange 510. The flexing wing cut outs 520 provide flexibility in the apparatus for comfort of one embodiment of the present invention.

FIG. 5C shows for illustrative purposes only shows an example of an oral device perspective front view of one embodiment of the present invention. FIG. 5C illustrates one example of the oral device 155 of FIG. 1 is the dentally stabilized platform apparatus 500. The tubular feature to allow the passage of ambient air through the apparatus 390 is seen through the opening extending to the rear of the apparatus. One section of the intra-oral flange 510 is to the side of the upper tooth channel 505. The lower tooth channel 505 terminates at the end of the flared rear wing 530 at the rear of the apparatus of one embodiment of the present invention.

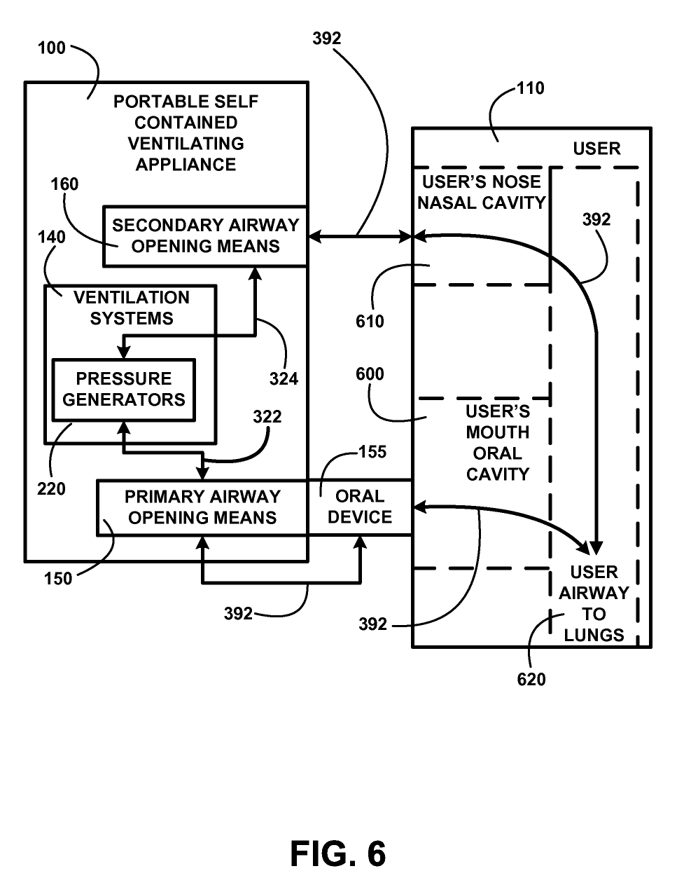

FIG. 6 shows for illustrative purposes only shows a flow diagram example of an installed portable self contained ventilating appliance of one embodiment of the present invention. FIG. 6 demonstrates by illustrating an example of the operating position on a user 110 and an example of a ventilating process using the portable self contained ventilating appliance 100 and the oral device 155 which is inserted into the user's mouth oral cavity 600. The portable self contained ventilating appliance 100 ventilation system 140 include the pressure generators 220. The pressure generators 220 deliver pressurized air to or draw air from the oral cavity 322 through the primary airway opening means 150. The passage of ambient or treated air 392 flows through the oral device 155 passing through a user's mouth oral cavity 600. The passage of ambient or treated air 392 continues to a user airway to lungs 620 of one embodiment of the present invention.

The secondary airway opening means 160 is positioned at the opening of a user's nose nasal cavity 610. The pressure generators 220 deliver pressurized air to or draw air from the nasal cavity 324 and acts as the secondary airway opening means 160. The passage of ambient or treated air 392 flows through the user's nose nasal cavity 610 and continues to the user airway to lungs 620 of one embodiment of the present invention.

### Example of Parallel Oral and Nasal Ventilation:

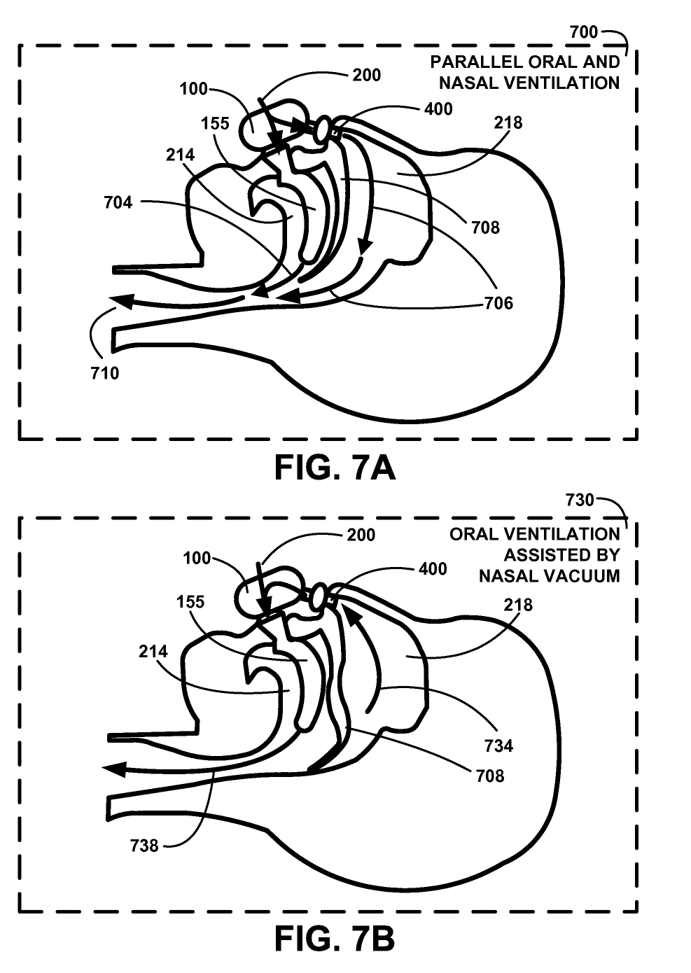

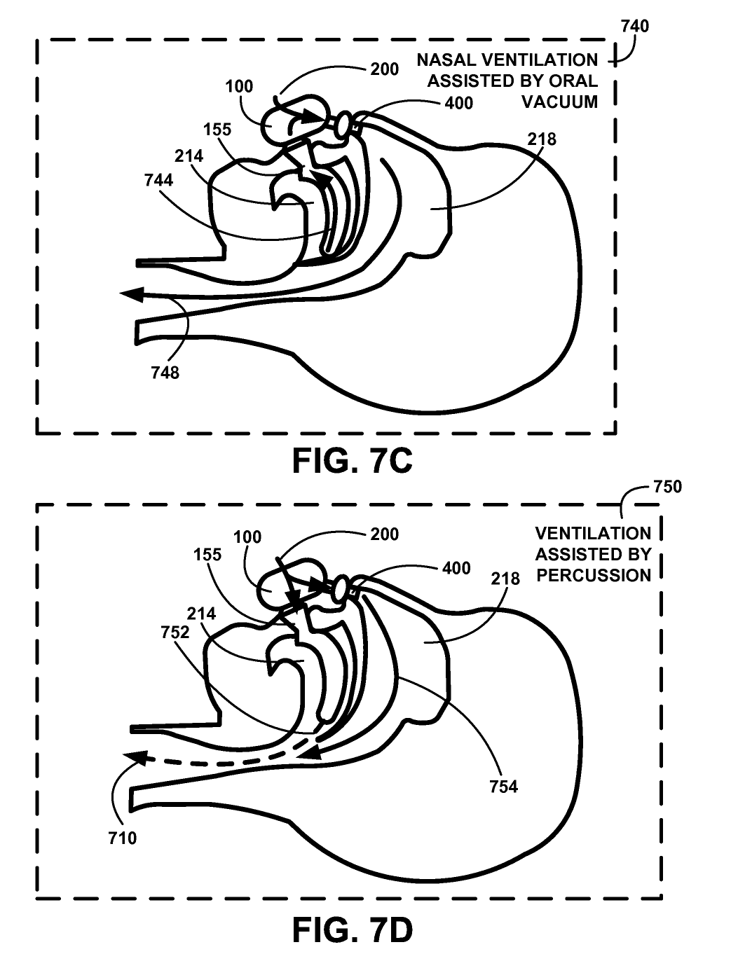

FIG. 7A shows for illustrative purposes only an example of parallel oral and nasal ventilation of one embodiment of the present invention. FIG. 7A illustrates an example of the present invention in an operation wherein parallel oral and nasal ventilation 700 are being performed by the portable self contained ventilating appliance 100 and the oral device 155 in concert. Ambient atmosphere 200 is drawn, filtered and forced through the oral device 155 and nasal airway connector 400. This creates positive pressure oral air flow to lungs 704 that is applied to the user's oral cavity 214. Air forced through the nasal airway connector 400 creates positive pressure nasal air flow 706 that is applied to the user's nasal cavity 218 above the upper palette area 708 of the mouth. The user is assisted by the combination air flow 710 to the lungs of one embodiment of the present invention.

### Example of Oral Ventilation Assisted By Nasal Vacuum:

FIG. 7B shows for illustrative purposes only shows an example of oral ventilation assisted by nasal vacuum of one embodiment of the present invention. FIG. 7B illustrates an example of the present invention in an operation wherein oral ventilation assisted by nasal vacuum 730 is being performed by the portable self contained ventilating appliance 100 and the oral device 155. Air is drawn through the nasal airway connector 400 from the user's nasal cavity 218 to create negative pressure nasal cavity to close nasal airway 734. Ambient atmosphere 200 is drawn, filtered and forced through the oral device 155 to the user's oral cavity 214 thusly creating positive pressure oral air flow 738 to the lungs to assist the user in breathing through the mouth of one embodiment of the present invention.

### Example of Nasal Ventilation Assisted By Oral Vacuum:

FIG. 7C shows for illustrative purposes only shows an example of nasal ventilation assisted by oral vacuum of one embodiment of the present invention. FIG. 7C illustrates an example of the present invention in an operation wherein nasal ventilation assisted by oral vacuum 740 is performed by the portable self contained ventilating appliance 100 and the oral device 155. Air is drawn out of the user's oral cavity 214 through the oral device 155 creating negative pressure oral air flow opens nasal airway 744. Ambient atmosphere 200 is drawn, filtered and forced through the nasal airway connector 400 to the user's nasal cavity 218 to create positive pressure nasal air flow to lungs 748 to assist the user in breathing through the nose of one embodiment of the present invention.

### Example of Ventilation Assisted By Percussion:

FIG. 7D shows for illustrative purposes only shows an example of ventilation assisted by percussion of one embodiment of the present invention. FIG. 7D illustrates an example of the present invention in an operation wherein ventilation assisted by percussion 750 is performed by the portable self contained ventilating appliance 100 and the oral device 155. Ambient atmosphere 200 is drawn, filtered and forced through the nasal airway connector 400 to the user's nasal cavity 218 to create positive pressure nasal air flow to lungs 754. Air is forced through the oral device 155 to the user's oral cavity 214 using positive percussion pressure oral air flow to lungs 752. The portable self contained ventilating appliance 100 processes response data to adjust the timing of acoustic excitation during the inspiratory cycle to prevent blockage. The combination air flow to lungs 710 thusly assists the user in breathing of one embodiment of the present invention.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. A portable self contained ventilating appliance, comprising:
an interface device coupled to at least one of an oral cavity or nasal cavity of a user;
a ventilation device physically extending from the interface and configured to deliver pressurized air to the user through the interface; and
a sensor monitoring system coupled to the interface and the ventilation device and configured to monitor the user.

2. The portable self contained ventilating appliance of claim 1, further comprising a rechargeable power supply located internal to the ventilation device.

3. The portable self contained ventilating appliance of claim 1, further comprising a two way wireless telemetry system transmitting and receiving information and adjustments to internal appliance settings from external sources.

4. The portable self contained ventilating appliance of claim 1, further comprising a treatment application system delivering medications or other treatments.

5. The portable self contained ventilating appliance of claim 1, wherein the ventilation system includes both primary and secondary air filter systems to filter incoming ambient air.

6. The portable self contained ventilating appliance of claim 1, wherein the ventilation system includes multiple pressure generators to create positive and negative pressure in the user oral and nasal cavities.

7. The portable self contained ventilating appliance of claim 1, wherein the ventilation system includes an air flow pressure controller to regulate the operation of the pressure generators.

8. The portable self contained ventilating appliance of claim 1, wherein the ventilation system includes a humidity controller of inspired air to regulate the amount of humidity being delivered to the user.

9. The portable self contained ventilating appliance of claim 1, wherein the ventilation system includes a system to introduce therapeutic agents and physiological agents into the air being delivered to the user.

10. The portable self contained ventilating appliance of claim 1, wherein the sensor monitoring system includes a respiratory processes monitor for assessing a user's respiration.

11. The portable self contained ventilating appliance of claim 1, wherein the sensor monitoring system includes a processing system for the physiological monitoring systems to assess the user's response to treatment.

12. The portable self contained ventilating appliance of claim 1, wherein the internal processing and control system includes a processor and controller for regulating the operations of the appliance systems.

13. The portable self contained ventilating appliance of claim 1, wherein the internal processing and control system includes plural user response parameters to measure user response information and determine appropriate settings adjustments automatically.

14. A portable appliance for treating sleep disorders, comprising:
an oral interface configured to be received by an oral cavity of a user;
a nasal device configured to create a rhinal seal of nose nostrils of the user;
a battery operated ventilation device physically extending from the oral interface and coupled to the oral device and the nasal device, wherein the ventilation device is configured to deliver air pressure to the oral cavity and nasal cavity of the user;
a sensor system coupled to the oral interface and configured to sense and monitor physiological occurrences from the oral cavity of the user; and
a wireless communications system internal to the battery operated ventilation device and configured to wirelessly communicate with a computer control system.

15. The portable appliance of claim 14, wherein the oral appliance includes a mandibular advancement device for adjusting a mandible of the user.

16. The portable appliance of claim 14, further comprising an air flow pressure controller and an internal processing and control system, wherein the internal processing and control system are configured to transmit setting adjustments to the air flow pressure controller to regulate ventilating delivery operations.

17. A portable self contained ventilating appliance, comprising:
an interface device coupled to at least one of an oral cavity or nasal cavity of a user;
a ventilation device physically extending from the interface and configured to deliver pressurized air to the user through the interface;
a sensor monitoring system coupled to the interface and the ventilation device and configured to monitor the user; and
a controller internal to the ventilation device and configured to make setting adjustments and air pressure delivery changes the user's oral and nasal cavities in response to the sensor monitoring system.

18. The portable self contained ventilating appliance of claim 17, wherein the sensor monitoring system is configured to detect an airway collapse of the oral cavity.

19. The portable self contained ventilating appliance of claim 17, wherein negative pressure is applied to the interface from the ventilation device.

20. The portable self contained ventilating appliance of claim 17, further comprising a treatment application system configured to retain a supply of various therapeutic agents and physiological agents and configured to administer accurate doses of each agent automatically according to internal instructions determined by the sensor and ventilation device.
